(19) United States
(12) Patent Application Publication
(10) Pub. No.: US 2017/0216181 A1
(43) Pub. Date: Aug. 3, 2017

(54) ANHYDROUS COMPOSITION BASED ON STYLING AND/OR SEBUM-ABSORBING POWDER AND ON PARTICLES ENCAPSULATING A BENEFICIAL AGENT

(71) Applicant: L'OREAL, Paris (FR)

(72) Inventors: Lionel AUBERT, Asnieres sur Oise (FR); Nathalie BEAU, Eragny-sur-Oise (FR)

(21) Appl. No.: 15/324,804

(22) PCT Filed: Jul. 9, 2015
(86) PCT No.: PCT/FR2015/051896

§ 371 Date: Jan. 9, 2017

(30) Foreign Application Priority Data
Jul. 9, 2014 (FR) 1456629
Jul. 9, 2014 (FR) 1456632
Oct. 29, 2014 (FR) 1460404

(51) Int. Cl. A61K 8/73; A61K 8/02; A61Q 5/06; A61K 8/31; A61Q 5/02; A61K 8/11; A61K 8/19

(52) U.S. Cl. CPC A61K 8/732; A61K 8/11; A61K 8/0279; A61K 8/022; A61K 8/19; A61K 8/31; A61Q 5/02; A61Q 5/06; A61K 2800/31

(57) ABSTRACT

A subject of the present invention is an anhydrous composition comprising:

a) at least particles encapsulating at least one beneficial agent, the particles being able to release the beneficial agent in the presence of water;
b) at least one sebum-absorbing powder with a sebum uptake of greater than or equal to 35 ml/100 g, and/or at least one styling powder other than the particles encapsulating at least one beneficial agent;
c) optionally a propellant.

A subject of the present invention is also to a process for the dry-washing and cosmetic treatment of keratin materials, comprising a step of applying the composition to the hair.

In the field of washing keratin materials, dry shampoos have existed for many years, either in a powdery form or in aerosol form. They enable excess sebum to be removed quickly without wetting the head of hair. They act by taking up sebum by absorption by means of powders chosen for their sebum-absorbing qualities.

The powders used may be of mineral, organic or synthetic origin and may be wheat, rice and maize starch derivatives.

In practice, the proposed dry shampoos are not entirely satisfactory. In particular, the clean feel perceived by users does not persist throughout the day, especially after physical activity.

There is thus a need to develop a novel dry shampoo composition which both offers optimum cleansing activity and gives the head of hair volume, with a clean feel which lasts over time.

The applicant has found, surprisingly and advantageously, that the use of at least one sebum-absorbing powder and/or of a styling powder in combination with particles encapsulating at least one beneficial agent which can release the beneficial agent in the presence of water made it possible to offer the cleansing properties expected of a dry shampoo and also styling properties such as giving the head of hair volume and body with a clean, fresh feel which persists over time.

The composition according to the invention especially enables the controlled release of the beneficial agent as a function of the presence of water/moisture on the scalp and/or in the hair, in particular following physical activity.

Moreover, the composition according to the invention is stable, in particular the capsules of beneficial agent are not degraded within the composition.

When the composition is in aerosol form, the composition may be satisfactorily dispensed.

Moreover, the composition of the invention leaves less white residue than the products conventionally used.

Finally, the composition of the invention is well tolerated by the scalp.

The subject of the invention is thus an anhydrous composition comprising:

a) at least particles encapsulating at least one beneficial agent, the particles being able to release the beneficial agent in the presence of water;
b) at least one sebum-absorbing powder having a sebum uptake of greater than or equal to 35 ml/100 g and/or at least one styling powder other than the particles encapsulating at least one beneficial agent;
c) optionally a propellant.

For the purposes of the present invention, the term “anhydrous composition” means a composition having a water content of less than 5% by weight, preferably less than 2% by weight, and/or a composition which does not contain any added water, that is to say that the water which may be present in the composition according to the invention is more particularly bound water, such as the water of crystallization of salts, or traces of water absorbed by the starting materials used in the production of the compositions according to the invention.

According to the invention, the composition may comprise one or more sebum-absorbing powders with a sebum uptake of greater than or equal to 35 ml/100 g,

For the purposes of the present invention, the term “sebum-absorbing powder” means a powder that is capable of absorbing and/or adsorbing sebum, which has a sebum uptake of greater than or equal to 35 ml/100 g.

The sebum uptake corresponds to the amount of sebum absorbed and/or adsorbed by the powder. It is expressed in ml of sebum per 100 g of powder and is measured using the method for determining the oil uptake of a powder described in standard NF T 30-022.

The oil uptake of a powder corresponds to the amount of sebum absorbed onto the available surface of the powder by measuring the “wet point” as indicated below.

The measuring method is as follows: an amount m (in grams) of between 0.5 and 5 grams of powder is placed on a glass plate, the amount depending on the density of the powder, followed by dropwise addition of artificial sebum having the following composition:

| | | |
|---|---|---|
| Triolein | 29% | by weight |
| Oleic acid | 28.5% | by weight |
| Oleyl oleate | 18.5% | by weight |
| Squalene | 14% | by weight |
| Cholesterol | 7% | by weight |
| Cholesteryl palmitate | 3% | by weight |

After addition of 4 to 5 drops of artificial sebum, the artificial sebum is incorporated into the powder using a spatula, and addition of the artificial sebum is continued until conglomerates of artificial sebum and powder have formed. At this point, the artificial sebum is added one drop at a time and the mixture is then triturated with the spatula.

The addition of artificial sebum is stopped when a firm, smooth paste is obtained. This paste must be able to be spread on the glass plate without cracking or forming lumps. The volume Vs, in ml, of artificial sebum used is then noted.

The sebum uptake corresponds to the ratio Vs/m.

The sebum-absorbing powder(s) used in the aerosol device of the invention have a sebum uptake preferably ranging from 35 to 1000 ml/100 g and even better still from 35 to 800 ml/100 g.

Advantageously, the sebum-absorbing particle may have a BET specific surface area of greater than or equal to 150 m²/g, preferably greater than or equal to 300 m²/g, better still greater than 500 m²/g and preferentially greater than 600 m²/g, and especially less than 1500 m²/g.

The BET specific surface area is determined according to the BET (Brunauer-Emmett-Teller) method described in the Journal of the American Chemical Society, vol. 60, page 309, February 1938, which corresponds to international standard ISO 5794/1 (appendix D). The BET specific surface area corresponds to the total specific surface area (thus including micropores) of the particle and especially of the powder.

The sebum-absorbing powder may be a mineral powder or an organic powder.

More precisely, the sebum-absorbing powder may be chosen from:

starches,

calcium silicates,

perlites,

zeolites,

polylactic acids,

silicas,

polyamide (nylon®) powders,

powders of acrylic polymers, in particular of polymethyl methacrylate, of poly(methyl methacrylate/ethylene glycol dimethacrylate), of poly(allyl methacrylate/ethylene glycol dimethacrylate) or of ethylene glycol dimethacrylate/lauryl methacrylate copolymer;

silicone elastomer powders, obtained especially by polymerization of organopolysiloxane containing at least two hydrogen atoms each bonded to a silicon atom and of an organopolysiloxane comprising at least two ethylenically unsaturated groups (especially two vinyl groups) in the presence of a platinum catalyst; and

mixtures thereof.

The sebum-absorbing powder may be a powder coated with a hydrophobic treatment agent.

The hydrophobic treatment agent can be chosen from fatty acids, such as stearic acid; metal soaps, such as aluminium dimyristate or the aluminium salt of hydrogenated tallow glutamate; amino acids; N-acylamino acids or their salts; lecithin, isopropyl-triisostearyl titanate, and mixtures thereof.

The N-acylamino acids can comprise an acyl group containing from 8 to 22 carbon atoms, for instance a 2-ethylhexanoyl, caproyl, lauroyl, myristoyl, palmitoyl, stearoyl or cocoyl group. The salts of these compounds can be the aluminium, magnesium, calcium, zirconium, zinc, sodium or potassium salts. The amino acid can, for example, be lysine, glutamic acid or alanine.

The term “alkyl” mentioned in the compounds cited above denotes in particular an alkyl group containing from 1 to 30 carbon atoms and preferably containing from 5 to 16 carbon atoms.

The starches which can be used in the present invention are, for example, maize starch, potato starch, tapioca starch, rice starch, wheat starch and cassava starch.

The starches may or may not be modified.

A modified starch is a starch which has been modified by processes known to a person skilled in the art, such as, for example, esterification, etherification, oxidation, acid hydrolysis, crosslinking or enzymatic conversion.

Non-limiting examples of modified starch comprise aluminium starch octenylsuccinate, sodium starch octenylsuccinate, calcium starch octenylsuccinate, distarch phosphate, hydroxyethyl starch phosphate, hydroxypropyl starch phosphate, sodium carboxymethyl starch and sodium starch glycolate.

In one particular embodiment, the starch is a starch octenylsuccinate, in particular an aluminium starch octenylsuccinate, the starch being from maize, wheat or rice. Mention may be made especially of the product sold by Akzo Nobel under the name Dry Flo Plus.

Preferably, the calcium silicates used as sebum-absorbing powder exhibit a sebum uptake of greater than 200 ml/100 g, better still between 400 ml/100 g and 600 ml/100 g and more preferentially of approximately 475 ml/100 g.

The specific surface area (BET) preferably ranges from approximately 150 m²/g to 600 m²/g, better still from 300 m²/g to 600 m²/g and even more preferentially from 310 m²/g to 350 m²/g.

The size of the silicate particles is preferably less than 20 micrometres.

These calcium silicates are generally prepared by reaction of reactive silica with an alkaline-earth metal reagent, preferably an alkaline-earth metal oxide or hydroxide, and a source of aluminium such as sodium aluminate or alumina. As the final properties of the silicate depend on the reactivity of the silica, the preferred source of silica is the reaction product of a soluble silicate, such as sodium silicate, and of a mineral acid, such as sulfuric acid. Suitable amorphous synthetic alkaline-earth metal silicates are manufactured by the company JM Huber Corporation and are sold under the Hubersorb® names. Methods for preparing these silicas are disclosed in greater detail in patent U.S. Pat. No. 4,557,916. Other suitable silicates are available from JM Huber Corporation, such as the sodium aluminosilicate sold under the Zeolexg brand name and the sodium magnesium aluminosilicate sold under the Hydrex® brand name.

Sebum-absorbing powders that may also be used include perlites, which are generally aluminosilicates of volcanic origin and which have the following composition:

70.0-75.0% by weight of silica SiO₂

12.0-15.0% by weight of aluminium oxide Al₂O₃

3.0-5.0% of sodium oxide Na₂O

3.0-5.0% of potassium oxide K₂O

0.5-2% of iron oxide Fe₂O₃

0.2-0.7% of magnesium oxide MgO

0.5-1.5% of calcium oxide CaO

0.05-0.15% of titanium oxide TiO₂.

Examples of zeolites that may especially be mentioned include sodium or potassium aluminosilicate compounds such as the product provided by Zeochem under the name Xmol.

The polylactic acids which can be used in the present invention are in particular Accurel EP600 from Akzo Nobel or the product provided under the name Lactic Acid Polymer 9105 by Dajac Labs.

Mention may be made, as silica powder, of:
the porous silica microspheres sold under the name Silica Beads SB-700 by the company Miyoshi; Sunsphere® H51 or Sunsphere® H33 by the company Asahi Glass;
the polydimethylsiloxane-coated amorphous silica microspheres sold under the name SA Sunsphere® H33 or SA Sunsphere® H53 by the company Asahi Glass.

Mention may be made, as nylon powder, of the nylon powder sold under the name Orgasol® 4000 by Atochem.

Mention may be made, as acrylic polymer powder, of:
the polymethyl methacrylate powders sold under the name Covabead® LH85 by the company Wackherr;
the polymethyl methacrylate/ethylene glycol dimethacrylate powders sold under the name Dow Corning 5640 Microsponge® Skin Oil Adsorber by the company Dow Corning; Ganzpearl® GMP-0820 by the company Ganz Chemical;
the poly(allyl methacrylate/ethylene glycol dimethacrylate) powders sold under the name Poly-Pore® L200 or Poly-Pore® E200 by Amcol Health and Beauty Solutions Inc.; these powders have in particular a sebum uptake of greater than or equal to 1 ml/g, better still ranging from 1 ml/g to 20 ml/g;
the ethylene glycol dimethacrylate/lauryl methacrylate copolymer powders sold under the name Polytrap® 6603 from the company Dow Corning.

Mention may be made, as silicone elastomer powder, of the powders sold under the names Trefil® Powder E-505C and Trefil® Powder E-506C by the company Dow Corning.

Preferably, the sebum-absorbing powder is chosen from modified starches such as starch octenylsuccinates and in particular aluminium starch octenylsuccinates, perlite, polylactic acids and zeolites, and better still from starch octenylsuccinates.

The sebum-absorbing powder(s) may be present in an amount preferably ranging from 0.1% to 99% by weight, even better still from 1% to 90% by weight and even more preferentially from 2% to 80% by weight, relative to the total weight of the composition.

The composition may also comprise one or more styling powders other than the sebum-absorbing powders.

The term “styling powder” means a powder consisting of one or more water-insoluble mineral compound(s) having a capacity for shaping the head of hair or for the durability of this shaping.

The water-insoluble mineral compound(s) is or are chosen from metal carbonates, oxides and sulfates, and silicates containing magnesium.

For the purposes of the present invention, the term “water-insoluble” means a compound of which the solubility at spontaneous pH in water at 25° C. and at atmospheric pressure is less than 0.1%.

Examples that may more particularly be mentioned include the carbonates, oxides and sulfates of alkaline-earth metals such as beryllium, magnesium, calcium, strontium, barium and radium, better still magnesium and calcium; the oxides, sulfates and carbonates of aluminium, gallium and indium; and silicates containing magnesium, particularly those containing an amount of magnesium of greater than 10% by weight (on a dry basis) as expressed in terms of magnesium oxide, such as Li—Mg—Na silicates, for instance Laponite XLG, which is provided by the company Rockwood.

More preferentially, use will be made of calcium carbonate, magnesium carbonate, alumina, barium sulfate and/or magnesium oxide, and even better still calcium carbonate. Preferably, these compounds have a mean particle size of from 20 to 50 μm, as water-insoluble mineral compound(s).

The water-insoluble mineral compound(s) may be present in an amount ranging from 0.1% to 99% by weight, even better still from 1% to 90% by weight and even more preferentially from 2% to 80% by weight, relative to the total weight of the composition.

According to the invention, the composition comprises particles encapsulating at least one beneficial agent, the particles being able to release the beneficial agent in the presence of water.

Within the meaning of the invention, the term “beneficial agent” means any compound present in a consumer product, which produces a beneficial effect perceived by the consumer during the use thereof and/or obtained on the consumer product itself, said beneficial effect possibly being a sensorial improvement or an especially visual and/or olfactory and/or tactile modification, an improvement in comfort and/or ease of application, an aesthetic effect, a hygienic effect, a clean feel or a curative and/or prophylactic effect.

The particles encapsulating at least one beneficial agent used in the composition according to the invention are able to release the beneficial agent in the presence of water.

At least one beneficial agent is immobilized, captured and/or encapsulated in the matrix of an encapsulation or trapping system; said beneficial agent being released outwards as the encapsulation or trapping system deteriorates, when it degrades on contact with water.

The particles encapsulating at least one beneficial agent used in the composition according to the invention may comprise a core containing at least one beneficial agent and a casing surrounding the core. These particles are hollow (vesicular), and are referred to as microcapsules.

The term “particles comprising a core containing at least one beneficial agent” means a particle comprising at least one beneficial agent immobilized, captured and/or encapsulated in the matrix of an encapsulation or trapping system; said beneficial agent being released outwards as the encapsulation or trapping system deteriorates, when it degrades on contact with a medium with which it will react, or under the effect of a stimulus such as a supply of water.

Alternatively, the particles encapsulating at least one beneficial agent used in the composition according to the invention may comprise a porous matrix, the beneficial agent being contained in the pores of the matrix. These particles are solid (matrix-based) and are referred to as microspheres.

The particles encapsulating at least one beneficial agent used in the composition according to the invention may comprise a material chosen from polyvinyl acetate, polyvinyl alcohol, (meth)acrylic (co)polymers, polyolefins, gelatine, chitosans, dextrins, natural or modified starches, plant gums, pectins, xanthans, alginates, carrageenans or cellulose derivatives such as carboxymethylcellulose, methylcellulose or hydroxyethylcellulose, preferably chosen from natural or modified starches.

When the particles encapsulating at least one beneficial agent comprise a core and a shell surrounding the core, the shell may comprise at least one material chosen from polyvinyl acetate, polyvinyl alcohol, (meth)acrylic (co)polymers, polyolefins, gelatin, chitosans, dextrins, natural or modified starches, plant gums, pectins, xanthans, alginates, carrageenans or cellulose derivatives such as carboxymethylcellulose, methylcellulose or hydroxyethylcellulose.

When the particles encapsulating at least one beneficial agent comprise a porous matrix, the porous matrix may comprise at least one material chosen from polyvinyl acetate, polyvinyl alcohol, (meth)acrylic copolymers, polyolefins, gelatine, chitosans, dextrins, natural or modified starches, plant gums, pectins, xanthans, alginates, carrageenans or cellulose derivatives such as carboxymethylcellulose, methylcellulose or hydroxyethylcellulose.

According to a preferred embodiment, the particles encapsulating at least one beneficial agent comprise a core and a casing comprising at least one material chosen from natural or modified starches.

The particles encapsulating at least one beneficial agent used in the composition according to the invention may be spherical. The term “spherical” means that the particle has a sphericity index, that is to say that the ratio between its largest diameter and its smallest diameter is less than 1.2. In this case, such particles are generally referred to as “capsules”.

The spherical particles in accordance with the present invention preferably have a number-average diameter ranging from 1 to 30 μm, more preferentially ranging from 2 to 15 μm and better still from 5 to 10 μm and a volume-average diameter ranging from 5 to 150 μm, preferably ranging from 10 to 100 μm and better still from 20 to 80 μm.

The term “average diameter” of the particles means the D[4,3] or D[1,0] parameters measured dry by laser diffraction, for example by means of a Microtrac S3500 particle size analyser, the results being expressed in the form of volume-based or number-based particle size distributions, respectively, leading to the average diameter.

The beneficial agent contained in the capsules of the invention is as defined above.

The beneficial agent contained in the capsules of the invention preferably produces a beneficial effect chosen from an olfactory modification, a clean feel, a refreshing feel, an antimicrobial activity or a soothing activity. The encapsulated beneficial agent may be chosen from fragrancing substances, surfactants, antimicrobial agents, refreshing agents such as menthol or mint, and soothing agents.

The encapsulated beneficial agent is preferably a fragrancing substance.

The term “fragrancing substance” means any ingredient liable to release a pleasant odour.

The fragrances are compositions especially containing raw materials (generally referred to as perfume ingredients) which are described in S. Arctander, Perfume and Flavor Chemicals (Montclair, N.J., 1969), in S. Arctander, Perfume and Flavor Material of Natural Origin (Elizabeth, N.J., 1960) and in “Flavor and Fragrance Materials—1991”, Allured Publishing Co, Wheaton, Ill.

These may be synthetic products or natural products, such as essential oils, absolutes, resinoids, resins, concretes, and/or synthetic products (terpene or sesquiterpene hydrocarbons, alcohols, phenols, aldehydes, ketones, ethers, acids, esters, nitriles, peroxides, which are saturated or unsaturated, aliphatic or cyclic).

According to the definition given in international standard ISO 9235 and adopted by the European Pharmacopoeia Commission, an essential oil is an odoriferous product generally of complex composition, obtained from a botanically defined plant raw material, either by steam entrainment, or by dry distillation, or via an appropriate mechanical process without heating. The essential oil is generally separated from the aqueous phase via a physical process which does not result in any significant change in the composition.

Among the essential oils that may be used according to the invention, mention may be made of those obtained from plants belonging to the following botanical families:

Abietaceae or Pinaceae: conifers; Amaryllidaceae; Anacardiaceae; Anonaceae: ylang ylang; Apiaceae (for example Umbelliferae): dill, angelica, coriander, sea fennel, carrot, parsley; Araceae; Aristolochiaceae; Asteraceae: yarrow, artemisia, camomile, helichrysum; Betulaceae; Brassicaceae; Burseraceae: frankincense; Caryophyllaceae; Canellaceae; Cesalpiniaceae: copaifera (copaiba balsam); Chenopodaceae; Cistaceae: rock rose; Cyperaceae; Dipterocarpaceae; Ericaceae: gaultheria (wintergreen); Euphorbiaceae; Fabaceae; Geraniaceae: geranium; Guttiferae; Hamamelidaceae; Hernandiaceae; Hypericaceae: St-John's wort; Iridaceae; Juglandaceae; Lamiaceae: thyme, oregano, monarda, savory, basil, marjorams, mints, patchouli, lavenders, sages, catnip, rosemary, hyssop, balm; Lauraceae: ravensara, sweet bay, rosewood, cinnamon, litsea; Liliaceae: garlic, lily, lily of the valley, hyacinth, daffodil, etc.; Magnoliaceae: magnolia; Malvaceae; Meliaceae; Monimiaceae; Moraceae: hemp, hop; Myricaceae; Myristicaceae: nutmeg; Myrtaceae: eucalyptus, tea tree, paperbark tree, cajuput, backhousia, clove, myrtle; Oleaceae; Piperaceae: pepper; Pittosporaceae; Poaceae: lemon balm, lemongrass, vetiver; Polygonaceae; Renonculaceae; Rosaceae: roses; Rubiaceae; Rutaceae: all citrus plants; Salicaceae; Santalaceae: sandalwood; Saxifragaceae; Schisandraceae; Styracaceae: benzoin; Thymelaceae: agarwood; Tilliaceae; Valerianaceae: valerian, spikenard; Verbenaceae: lantana, verbena; Violaceae; Zingiberaceae: galangal, turmeric, cardamom, ginger; Zygophyllaceae.

Mention may also be made of the essential oils extracted from flowers (lily, lavender, rose, jasmine, ylang ylang, neroli), from stems and leaves (patchouli, geranium, petitgrain), from fruit (raspberry, peach, coriander, aniseed, cumin, juniper), from fruit peel (bergamot, lemon, orange, grapefruit), from roots (angelica, celery, cardamom, iris, sweet flag, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benzoin, myrrh, olibanum, opopanax).

Examples of fragrancing substances are especially: geraniol, geranyl acetate, farnesol, borneol, bornyl acetate, linalool, linalyl acetate, linalyl propionate, linalyl butyrate, tetrahydrolinalool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, cis-3-hexenyl acetate, vetiveryl acetate, ethyl acetate, butyl acetate, hexyl acetate, decyl acetate, isoamyl acetate, stearyl acetate, allyl heptanoate, vetiverol, alpha-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, allyl 3-cyclohexylpropionate, ethyl-6-(acetyloxy) hexanoate, allyl caproate, ethyl 2-methyl butyrate, methyl dihydrojasmonate, hexyl salicylate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranyl acetone, n-decanal, n-dodecanal, anisyl propanal, 9-decen-1-ol, cis-3-hexanol, 2-isobutyl-4-methyltetrahydropyran-4-ol, 3-methyl-5-phenyl-1-pentanol, 3a,6,6,9a-Tetramethyldodecahydronaphtho[2,1-b]furan, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropin, coumarin, eugenol, vanillin, diphenyl ether, citral, citronellal, hydroxycitronellal, hexylcinnamal, 2,4-dimethylcyclohex-3-ene-1-carbaldehyde, 2,6-dimethylhept-5-enal, α,α-dimethyl-p-ethylphenylpropanal, 1,3-benzodioxole-5-carboxaldehye, limonene, damascone, decalactone, nonalactone, 6,6-dimethoxy-2,5,5-trimethylhex-2-ene, 2,4,4,7-tetramethyloct-6-en-3-one, 1-(5,5-dimethyl-1-cyclohexenyl)pent-4-en-1-one, methylheptenone, 4-(cyclopropylmethyl)-phenylmethyl ether, 2-methyl-6-methylideneoct-7-en-2-ol, rose oxide, 1-(1,2,3,4,5,6,7,8-octahydro-2,3,8,8-tetramethyl-2-naphthyl)ethan-1-one, 2-acetonaphthone, 2-isopropyl-5-methylcyclohexanone, ionones, methylionones, isomethylionones, solanone, irones, cis-3-hexenol and esters thereof, indane musks, tetralin musks, isochroman musks, macrocyclic ketones, macrolactone musks, aliphatic musks, ethylene brassylate, rose essence, and mixtures thereof.

The particles according to the invention containing the beneficial agent preferably represent from 0.01 to 10% by weight, preferably from 0.05 to 5% by weight and better still from 0.1 to 3% by weight of the total weight of the composition.

The particles encapsulating at least one beneficial agent used in the composition according to the invention may be obtained by conventional encapsulation techniques. In particular, they may be obtained according to the process described in the patent U.S. Pat. No. 5,508,259. Especially, an emulsion is formed from a solid film-forming substrate chosen from polyvinyl acetate, polyvinyl alcohol, (meth)acrylic (co)polymers, polyolefins, gelatine, chitosans, dextrins, natural or modified starches, plant gums, pectins, xanthans, alginates, carrageenans or else cellulose derivatives such as, for example, carboxymethylcellulose, methylcellulose or hydroxyethylcellulose, in combination with an emulsifier and a mixture of perfume ingredients. The emulsion is then dehydrated by a conventional spray-drying process, which consists, as described in example 1 of patent U.S. Pat. No. 5,508,259, in spraying it in fine droplets from a spray with a flow rate of 50 kg/h and a pressure of 0.45 bars, in contact with a stream of air of 320 m³/h heated to 350° C. in order to evaporate the water, which makes it possible to obtain a fine powder with a particle diameter of between 20 and 80 microns and containing 20% by weight of fragrance.

By way of particles encapsulating at least one beneficial agent, mention may be made of the APPLE BLOSSOM CAPS 01 capsules sold by Firmenich, the EMERALD FIRCAPS capsules sold by Firmenich, the TECNI MIX PLUS capsules sold by Mane, the INCAP 50 Green Boost, INCAP 50 Clean and INCAP 50 Care capsules sold by Symrise.

As indicated previously, the composition according to the invention may also contain one or more propellants.

The propellant which may be used in the composition according to the invention may be chosen from air, nitrogen, carbon dioxide, dimethyl ether, volatile hydrocarbons such as, in particular, C₃-C₅ alkanes, chlorinated and/or fluorinated hydrocarbons such as 1,1-difluoroethane and mixtures thereof, preferably chosen from C₃-C₃ alkanes and preferably n-butane, propane, isobutane and mixtures thereof.

Mention may be made preferentially of C₃-C₃ alkanes and in particular propane n-butane and isobutane and mixtures thereof.

When they are present in the composition, the propellant(s) is or are present in an amount ranging from 10% to 95% by weight, even better still from 15% to 90% by weight and even more preferentially from 20% to 88% by weight, relative to the total weight of the composition.

The composition of the invention may also comprise one or more liquid fatty substances.

The term “fatty substance” means an organic compound that is insoluble in water at standard temperature (25° C.) and at atmospheric pressure (760 mmHg, i.e. 1.013×10⁵ Pa), i.e. with a solubility of less than 5%, preferably of less than 1% and even more preferentially of less than 0.1%.

The fatty substances are preferably non-siliceous, that is to say their structure does not contain a silicon atom. They generally have in their structure a hydrocarbon-based chain comprising at least 6 carbon atoms and not comprising any siloxane group.

In addition, the fatty substances are generally soluble in organic solvents under the same temperature and pressure conditions, for instance chloroform, ethanol, benzene, liquid petroleum jelly or decamethylcyclopentasiloxane.

The liquid fatty substances that may be used in the invention are liquid at ambient temperature (25° C.) and under atmospheric pressure (760 mmHg, i.e. 1.013×10⁵ Pa). They preferably have a viscosity of less than or equal to 2 Pa·s, better still less than or equal to 1 Pa·s, and even better still less than or equal to 0.1 Pa·s at a temperature of 25° C. and at a shear rate of 1 s⁻¹.

The liquid fatty substances that may be used in the composition according to the invention are generally not oxyalkylenated and preferably do not contain any carboxylic acid COOH functions.

The liquid fatty substances are preferably chosen from hydrocarbons, fatty alcohols, fatty esters and fatty ethers, and mixtures thereof.

Even more preferentially, they are chosen from hydrocarbons, fatty alcohols and fatty esters, and mixtures thereof.

The term “liquid hydrocarbon” means a hydrocarbon composed solely of carbon and hydrogen atoms, which is liquid at standard temperature (25° C.) and at atmospheric pressure (760 mmHg, i.e. 1.013×10⁵ Pa), which is especially of mineral or plant or synthetic origin.

More particularly, the liquid hydrocarbons are chosen from:
linear or branched, optionally cyclic, C₆-C₁₆ alkanes. Examples that may be mentioned include hexane, undecane, dodecane, tridecane, and isoparaffins, for instance isohexadecane, isododecane and isodecane,
linear or branched hydrocarbons of mineral, animal or synthetic origin with more than 16 carbon atoms, such as liquid paraffin or liquid petroleum jelly, polydecenes, hydrogenated polyisobutene such as the product sold under the brand name Parleam® by the company NOF Corporation, and squalane.

In one preferred variant, the liquid hydrocarbon(s) is or are chosen from liquid paraffin and liquid petroleum jelly.

The term “liquid fatty alcohol” means a non-glycerolated and non-oxyalkylenated fatty alcohol which is liquid at standard temperature (25° C.) and at atmospheric pressure (760 mmHg; i.e. 1.013×10⁵ Pa).

Preferably, the liquid fatty alcohols of the invention comprise from 8 to 30 carbon atoms.

The liquid fatty alcohols of the invention may be saturated or unsaturated.

The saturated liquid fatty alcohols are preferably branched. They may optionally comprise in their structure at least one aromatic or non-aromatic ring. They are preferably acyclic.

More particularly, the liquid saturated fatty alcohols of the invention are chosen from octyldodecanol, isostearyl alcohol and 2-hexyldecanol.

Octyldodecanol is most particularly preferred.

The unsaturated liquid fatty alcohols contain in their structure at least one double or triple bond, and preferably one or more double bonds. When several double bonds are present, there are preferably 2 or 3 of them, and they may be conjugated or unconjugated.

These unsaturated fatty alcohols may be linear or branched.

They may optionally comprise in their structure at least one aromatic or non-aromatic ring. They are preferably acyclic.

More particularly, the unsaturated liquid fatty alcohols of the invention are chosen from oleyl alcohol, linoleyl alcohol, linolenyl alcohol and undecylenyl alcohol.

Oleyl alcohol is most particularly preferred.

The term “liquid fatty ester” means an ester derived from a fatty acid and/or from a fatty alcohol, that is liquid at standard temperature (25° C.) and at atmospheric pressure (760 mmHg, i.e. 1.013×10⁵ Pa).

The esters are preferably liquid esters of saturated or unsaturated, linear or branched, C₁-C₂₆ aliphatic monoacids or polyacids and of saturated or unsaturated, linear or branched, C₁-C₂₆ aliphatic monoalcohols or polyalcohols, the total number of carbon atoms in the esters being greater than or equal to 10.

Preferably, for the esters of monoalcohols, at least one of the alcohol or of the acid from which the esters of the invention result is branched.

Among the monoesters of monoacids and of monoalcohols, mention may be made of ethyl isopropyl palmitates, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols and esters of mono-, di- or tricarboxylic acids and of C₄-C₂₆ non-sugar di-, tri-, tetra- or pentahydroxylated alcohols may also be used.

Mention may be made especially of diethyl sebacate, diisopropyl sebacate, bis(2-ethylhexyl) sebacate, diisopropyl adipate, di-n-propyl adipate, dioctyl adipate, bis(2-ethylhexyl) adipate, diisostearyl adipate, bis(2-ethylhexyl) maleate, triisopropyl citrate, triisocetyl citrate, triisostearyl citrate, glyceryl trilactate, glyceryl trioctanoate, trioctyldodecyl citrate, trioleyl citrate, neopentyl glycol diheptanoate, and diethylene glycol diisononanoate.

The composition may further comprise, as liquid fatty ester, sugar esters and diesters of C₆-C₃₀ fatty acids, preferably C₁₂-C₂₂ fatty acids. It is recalled that the term “sugar” means oxygen-bearing hydrocarbon-based compounds which contain a plurality of alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, and polyesters, and mixtures thereof.

These esters may be, for example, oleates, laurates, palmitates, myristates, behenates, cocoates, stearates, linoleates, linolenates, caprates or arachidonates, or mixtures thereof, such as, especially, oleopalmitate, oleostearate or palmitostearate mixed esters.

More particularly, use is made of monoesters and diesters and especially of sucrose, glucose or methylglucose mono- or dioleates, stearates, behenates, oleopalmitates, linoleates, linolenates or oleostearates.

An example that may be mentioned is the product sold under the name Glucate® DO by the company Amerchol, which is a methylglucose dioleate.

Finally, use may also be made of natural or synthetic glycerol esters of mono-, di- or triacids.

Among these, mention may be made of plant oils.

As oils of plant origin or synthetic triglycerides that may be used in the composition of the invention as liquid fatty esters, examples that may be mentioned include:
triglyceride oils of plant or synthetic origin, such as liquid fatty acid triglycerides containing from 6 to 30 carbon atoms, for instance heptanoic or octanoic acid triglycerides, or alternatively, for example, sunflower oil, maize oil, soya bean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, safflower oil, candlenut oil, camelina oil, tamanu oil, babassu oil and pracaxi oil, caprylic/capric acid triglycerides, for instance those sold by the company Stéarineries Dubois or those sold under the names Miglyol® 810, 812 and 818 by the company Dynamit Nobel, jojoba oil and shea butter oil.

Liquid fatty esters derived from monoalcohols will preferably be used as esters according to the invention.

Isopropyl myristate or isopropyl palmitate are particularly preferred.

The liquid fatty ethers are chosen from liquid dialkyl ethers such as dicaprylyl ether.

Preferably, the liquid fatty substances are chosen from linear or branched hydrocarbons of mineral, animal or synthetic origin and with more than 16 carbon atoms, branched or unsaturated fatty alcohols, fatty esters and triglyceride oils of plant origin.

More preferentially. the liquid fatty substances are chosen from liquid paraffin or liquid petroleum jelly, octyldodecanol, isostearyl alcohol, 2-hexyldecanol, oleyl alcohol, linoleyl alcohol, linolenyl alcohol and undecylenyl alcohol, ethyl and isopropyl palmitates, alkyl myristates such as isopropyl myristate or ethyl myristate, isocetyl stearate, 2-ethylhexyl isononanoate, isononyl isononanoate, isodecyl neopentanoate and isostearyl neopentanoate, and plant oils.

Even more preferentially, the liquid fatty substances are chosen from isopropyl palmitate and isopropyl myristate.

When they are present, the liquid fatty substance(s) is or are present in an amount ranging preferably from, for example, 0.05% to 20% by weight, better still from 0.1% to 10% by weight and even better still from 0.2% to 5% by weight, relative to the total weight of the composition.

The composition according to the invention may also contain one or more additives chosen from conditioning or fixing anionic, cationic, nonionic, amphoteric or zwitterionic polymers, fragrances, dyes, protective screening agents, acids, bases, nacres and glitter flakes.

These additives may be present in the composition according to the invention in an amount ranging from 0 to 20% by weight relative to the total weight of the composition.

A person skilled in the art will take care to choose these optional additives and their amounts so that they do not harm the properties of the compositions of the present invention.

According to one embodiment, the compositions in powder form in accordance with the invention may be packaged in a container comprising one or more dispensing orifices, of the salt shaker type.

According to another embodiment, the compositions in powder form in accordance with the invention may be packaged in an aerosol device composed of a container comprising the composition and a means for dispensing the said composition.

According to this embodiment, the container is pressurized. The container may be opaque or transparent. It can be made of glass, of polymer or of metal, optionally covered with a protective lacquer layer.

The container may therefore contain both the propellant(s) and the other ingredients of the composition, in a single compartment, or as a variant in two compartments. According to the latter variant, the container may consist of an outer aerosol can comprising an inner bag hermetically welded to a valve. The various ingredients of the composition are introduced into the inner bag and a propellant is introduced between the bag and the can at a sufficient pressure to make the product come out in the form of a spray.

The container is equipped at its top end with a valve that seals the system.

For example, it is possible to use a valve sold by the companies Precision, Coster, Seaquist and Lindal.

The device, packaged with such a valve, ensures the sealing of the system, and also the dispensing of the product from the container.

The present invention also relates to a process for the dry-washing and cosmetic treatment of the hair, comprising the application to the hair, preferably dry hair, of a composition as described above.

The present invention also relates to the use of the composition defined above for the dry-washing and cosmetic treatment of the hair.

### EXAMPLE

In the example that follows, all the amounts are indicated as weight percentages of product in unmodified form relative to the total weight of the composition.

The following compositions according to the invention are prepared starting from the compounds indicated in the tables below.

| | A |
|---|---|
| DRY FLO PLUS by National Starch (86% | 92.15 |
| Aluminium Starch Octenylsuccinate) | |
| Calcium carbonate (D50 = 35 μm)¹ | 1.45 |
| Hectorite modified with | 2.00 |
| distearyldimethylammonium chloride² | |
| Isopropyl myristate | 3.00 |
| Starch-based fragrance capsules³ | 1.40 |

¹Sold under the trade name Omyacare S60 by Omya
²Sold under the trade name Bentone 38 by Elementis
³Sold under the trade name Apple Blossom Caps by Firmenich

This composition A was introduced into a small bottle of salt-shaker type.

| | B |
|---|---|
| DRY FLO PLUS by National Starch (86% | 10.92 |
| Aluminium Starch Octenylsuccinate) | |
| Calcium carbonate (D50 = 35 μm)¹ | 2.18 |
| Hectorite modified with | 0.28 |
| distearyldimethylammonium chloride² | |
| Isopropyl myristate | 0.42 |
| Starch-based fragrance capsules³ | 0.19 |
| Isobutane | 86.00 |

¹Sold under the trade name Omyacare S60 by Omya
²Sold under the trade name Bentone 38 by Elementis
³Sold under the trade name Apple Blossom Caps by Firmenich

This composition B was introduced into an aerosol device. Said device is fitted with a valve conventionally used for aerosols. Composition A and composition B were applied to a greasy, dirty head of hair. The application is well tolerated by the scalp. A fresh feel is observed on application.

After drying, it is observed that the head of hair is visibly cleaner with little visible residues. Lifting of the roots is also observed, and also increased volume. In addition, an instant refreshing effect is observed, which lasts throughout the day. The fragrancing effect lasts throughout the day and is reinforced every time moisture appears.

1.-18. (canceled)

19. An anhydrous composition comprising:
a) at least one particle encapsulating at least one beneficial agent, wherein the particle is capable of releasing the beneficial agent in the presence of water;
b) at least one sebum-absorbing powder with a sebum uptake of greater than or equal to about 35 mL/100 g, and/or at least one styling powder other than the at least one particle encapsulating at least one beneficial agent; and
c) optionally, at least one propellant.

20. The composition according to claim 19, wherein the at least one sebum-absorbing powder is chosen from modified starches.

21. The composition according to claim 19, wherein the at least one sebum-absorbing powder is chosen from starch octenylsuccinates, aluminium starch octenylsuccinates, perlite, polylactic acids, or zeolites.

22. The composition according to claim 19, wherein the at least one sebum-absorbing powder is present in an amount ranging from about 0.1% to about 99% by weight, relative to the total weight of the composition.

23. The composition according to claim 19, wherein the at least one styling powder comprises at least one water-insoluble mineral compound other than the sebum-absorbing powders,
wherein said at least one water-insoluble mineral compound is chosen from metal carbonates, oxides, sulfates, or silicates containing magnesium.

24. The composition according to claim 23, wherein the at least one water-insoluble mineral compound is chosen from calcium carbonate, magnesium carbonate, alumina, barium sulfate, or magnesium oxide.

25. The composition according to claim 23, wherein the at least one water-insoluble mineral compound is present in an amount ranging from about 0.1% to about 99% by weight, relative to the total weight of the composition.

26. The composition according to claim 19, wherein the at least one particle encapsulating at least one beneficial agent comprises:
a core containing the at least one beneficial agent, and
a casing surrounding the core.

27. The composition according to claim 19, wherein the at least one particle encapsulating at least one beneficial agent comprises a porous matrix, wherein the at least one beneficial agent is contained in the pores of the matrix.

28. The composition according to claim 19, wherein the at least one particle encapsulating at least one beneficial agent comprises at least one material chosen from polyvinyl acetate, polyvinyl alcohol, (meth)acrylic (co)polymers, polyolefins, gelatine, chitosans, dextrins, natural or modified starches, plant gums, pectins, xanthans, alginates, or carrageenans or cellulose.

29. The composition according to claim 19, wherein the at least one beneficial agent is chosen from fragrancing substances, surfactants, antimicrobial agents, or soothing agents.

30. The composition according to claim 19, wherein the at least one particle encapsulating at least one beneficial agent is present in an amount ranging from about 0.01% to about 10% by weight, relative to the total weight of the composition.

31. The composition according to claim 19, wherein the at least one propellant is chosen from air, nitrogen, carbon dioxide, dimethyl ether, volatile hydrocarbons, chlorinated and/or fluorinated hydrocarbons, or mixtures thereof.

32. The composition according to claim 19, wherein the at least one propellant is present in the composition in an amount ranging from about 10% to 95% by weight, relative to the total weight of the composition.

33. The composition according to claim 19, further comprising at least one liquid fatty substance.

34. The composition according to claim 33, wherein the at least one liquid fatty substance is chosen from linear or branched hydrocarbons of mineral, animal or synthetic origin with more than 16 carbon atoms; branched or unsaturated fatty alcohols; fatty esters; or triglyceride oils of plant origin.

35. The composition according to claim 34, wherein the at least one liquid fatty substance is chosen from liquid paraffin or liquid petroleum jelly, octyldodecanol, isostearyl alcohol, 2-hexyldecanol, oleyl alcohol, linoleyl alcohol, linolenyl alcohol and undecylenyl alcohol, ethyl and isopropyl palmitates, alkyl myristates, isocetyl stearate, 2-ethylhexyl isononanoate, isononyl isononanoate, isodecyl neopentanoate and isostearyl neopentanoate, or plant oils.

36. An aerosol device consisting of a container, the container comprising:
a composition comprising:
a) at least one particle encapsulating at least one beneficial agent, wherein the particle is able to release the beneficial agent in the presence of water;
b) at least one sebum-absorbing powder with a sebum uptake of greater than or equal to about 35 mL/100 g, and/or at least one styling powder other than the at least one particle encapsulating at least one beneficial agent; and
c) optionally, at least one propellant; and
a means for dispensing the composition.

37. A process for dry-washing and cosmetic treatment of keratin materials, comprising applying to the hair a composition comprising:
a) at least one particle encapsulating at least one beneficial agent, wherein the particle is capable of releasing the beneficial agent in the presence of water;
b) at least one sebum-absorbing powder with a sebum uptake of greater than or equal to about 35 mL/100 g, and/or at least one styling powder other than the at least one particle encapsulating at least one beneficial agent; and
c) optionally, at least one propellant.
